(19) United States
(12) Patent Application Publication
(10) Pub. No.: US 2023/0279361 A1
(43) Pub. Date: Sep. 7, 2023

(54) METHOD FOR IN VITRO PROLIFERATING VIRUS BELONGING TO FAMILY CORONAVIRIDAE, METHOD FOR PRODUCING NEUTRALIZING ANTIBODY TO VIRUS BELONGING TO FAMILY CORONAVIRIDAE, AND METHOD FOR PRODUCING INFECTION MODEL OF SEVERE ACUTE RESPIRATORY SYNDROME CORONAVIRUS 2

(71) Applicant: NATIONAL UNIVERSITY CORPORATION SHIGA UNIVERSITY OF MEDICAL SCIENCE, Otsu-shi, Shiga (JP)

(72) Inventors: Yasushi ITOH, Otsu-shi, Shiga (JP); Hirohito ISHIGAKI, Otsu-shi, Shiga (JP); Misako NAKAYAMA, Otsu-shi, Shiga (JP); Yoshinori KITAGAWA, Otsu-shi, Shiga (JP)

(21) Appl. No.: 18/006,920

(22) PCT Filed: Jul. 27, 2021
(86) PCT No.: PCT/JP2021/027662

§ 371 Date: Jan. 26, 2023

(30) Foreign Application Priority Data
Jul. 27, 2020 (JP) 2020-126321

(51) Int. Cl. C12N 7/00; C12N 5/071; C07K 16/10

(52) U.S. Cl. CPC C12N 7/00; C12N 5/0686; C07K 16/10; C12N 2501/734; C12N 2770/20032; C12N 2770/20051

(57) ABSTRACT

The present invention addresses the problem of providing a more convenient method for in vitro proliferating a virus belonging to the family Coronaviridae at high proliferation accuracy. This problem is solved by an in vitro virus proliferation method, said method comprising a step for culturing kidney-derived cells, which have been contacted with the virus belonging to the family Coronaviridae, with a use of a cell culture medium to which a protease having an optimum pH of from 7 to 9 is added.

### TECHNICAL FIELD

Disclosed herein are a method for in vitro proliferating a virus belonging to the family Coronaviridae, a method for producing a neutralizing antibody to the virus belonging to the family Coronaviridae, and a method for producing an infection model of severe acute respiratory syndrome Coronavirus 2.

### BACKGROUND ART

Severe acute respiratory syndrome coronavirus 2 (SARS-CoV-2) infection (COVID-19) has spread worldwide since the latter half of 2019, and WHO had declared a pandemic on Mar. 11, 2020. The severity of infected individuals with the SARS-CoV-2 may vary, including for example, patients with various severity such as asymptomatic patients, patients with mild respiratory symptoms, and patients with acute respiratory distress syndrome (ARDS) requiring admission to an ICU. Since the number of patients infected with the SARS-CoV-2 continues to increase, there is an urgent need to clarify the pathogenesis in patients presenting with severe clinical manifestations of the disease and to develop vaccines and antivirals specific for the SARS-CoV-2.

Experimental animal models are indispensable for development of a therapeutic method. Non-Patent Literature 1 describes a SARS-CoV-2 infection model using a Macaca fascicularis. In addition, Non-Patent Literatures 2 to 5 describe SARS-CoV-2 infection models using Macaca mulattas. On the other hand, Patent Literature 1 describes a virus inspection method comprising an inoculation step for inoculating a dengue virus on a monkey belonging to the Cebidae and a detection step for detecting the dengue virus from the monkey.

### CITATION LIST

### Patent Literature

Patent Literature 1: JP5315494 (B2)

### Non Patent Literature

Non Patent Literature 1: Rockx et al., Science 368, 1012-1015 (2020) 29 May 2020
Non Patent Literature 2: Williamson et al., bioRxiv preprint doi: https://doi.org/10.1101/2020.04.15.043166. this version posted Apr. 15, 2020.
Non Patent Literature 3: Yu et al., Animal Model Exp Med. 2020; 3:93-97. Accepted: 12 Mar. 2020
Non Patent Literature 4: Munster et al., https://doi.org/10.1038/s41586-020-2324-7. Published online: 12 May 2020
Non Patent Literature 5: Gao et al., Science 369, 77-81 (2020) 3 Jul. 2020
Non Patent Literature 6: Matsuyama, PNAS Mar. 31, 2020 117 (13) 7001-7003; first published Mar. 12, 2020 https://doi.org/10.1073/pnas.2002589117

### SUMMARY OF INVENTION

### Technical Problem

Patent Literature 1 describes that the monkey belonging to the Cebidae is preferable as the monkey to be infected with the dengue virus, but the Macaca fascicularis is not preferable because infection with the dengue virus is difficult to be occurred in it. Thus, even if an animal classified as a primate is inoculated with a virus that is pathogenic to humans, whether or not infection is occurred depends on a family or species.

Moreover, when the SARS-CoV-2 invades a human body, the severity of symptoms caused by the SARS-CoV-2 may vary. The severity of the SARS-CoV-2 infection may also vary among patients, and it is unclear whether this variability depends on the SARS-CoV-2 or the patient, or both.

Therefore, in order to establish a therapeutic method for the SARS-CoV-2, it is necessary not only to establish an experimental animal model, but also to increase accuracy of an in vitro virus proliferation method and a virus titer measurement method, and thereby more accurately evaluate the nature of SARS-CoV-2 and a pathology thereof in a host using an animal model infected with the SARS-CoV-2. Non Patent Literature 6 describes that TMPRSS2, which is a serine protease present in a cell membrane of human respiratory cells, is forcibly expressed in Vero E6 cells and the forced expression strain is infected with the SARS-CoV-2 to allow them to be isolated and proliferate efficiently. However, this virus proliferation method requires the forced expression of the TMPRSS2 in cells.

Furthermore, from the viewpoint of prevention of the SARS-CoV-2 infection, development of the vaccine is strongly desired. Therefore, there is a need for an infection animal model that produces neutralizing antibodies to the virus belonging to the family Coronaviridae.

An object of the present invention is to provide a more convenient method for in vitro proliferating the virus belonging to the family Coronaviridae at high proliferation accuracy. In addition, another object of the present invention is to provide an animal model infected with the virus belonging to the family Coronaviridae that can produce the neutralizing antibodies.

### Solution to Problem

As a result of diligent research, the present inventors found that a use of a protease improved a proliferation of a virus belonging to the family Coronaviridae. In addition, they found that monkeys belonging to the Macaca produced a neutralizing antibody to the virus belonging to the family Coronaviridae.

The present invention has been achieved based on the above findings, and includes following aspects.
Item 1.

A method for in vitro proliferating a virus, comprising a step for culturing kidney-derived cells, which have been contacted with the virus belonging to the family Coronaviridae, with a use of a cell culture medium to which the protease having an optimum pH of from 7 to 9 is added.
Item 2.

The method according to item 1, wherein the virus is severe acute respiratory syndrome coronavirus 2.
Item 3.

The method according to item 1 or 2, wherein the protease is an endopeptidase.
Item 4.

The method according to item 3, wherein the protease is trypsin.
Item 5.

A method for producing the neutralizing antibody to a virus belonging to the family Coronaviridae comprising, a first step for inoculating the virus belonging to the family Coronaviridae and/or a part thereof serving as an antigen to a monkey belonging to the Macaca, a second step for collecting a blood sample from the monkey inoculated with the antigen, a third step for mixing the blood sample with the virus belonging to the family Coronaviridae, a fourth step for bringing the virus belonging to the family Coronaviridae mixed with the blood sample into contact with kidney-derived cells, a fifth step for culturing the kidney-derived cells, which have been contacted with the virus, with a use of a cell culture medium to which a protease having an optimum pH of from 7 to 9 is added, a sixth step for evaluating a condition of denaturation of the kidney-derived cells cultured in the fifth step, and a seventh step for determining that neutralizing antibodies are produced when there is no denaturation of the kidney-derived cells or degree of the denaturation of the kidney-derived cells is low in the evaluation of the sixth step.
Item 6.

The method for producing the neutralizing antibody according to item 5, wherein the monkey is a Macaca fascicularis.
Item 7.

The method for producing the neutralizing antibody according to item 5 or 6, wherein the virus is severe acute respiratory syndrome coronavirus 2.
Item 8.

A method for producing a neutralizing antibody to severe acute respiratory syndrome corona virus 2, comprising a step for inoculating the severe acute respiratory syndrome corona virus 2 and/or the part thereof serving as an antigen to a Macaca fascicularis.
Item 9.

A method for producing an infection model of severe acute respiratory syndrome coronavirus 2 comprising a step for culturing kidney-derived cells, which have been contacted with the severe acute respiratory syndrome coronavirus 2, with a use of a cell culture medium to which a protease having an optimum pH of from 7 to 9 is added, a step for collecting the severe acute respiratory syndrome coronavirus 2 from the culture of the kidney-derived cells, and a step for inoculating the collected severe acute respiratory syndrome corona virus 2 and/or a part thereof to a Macaca fascicularis.

### Advantageous Effects of Invention

It is possible to provide the more convenient method for in vitro proliferating the virus belonging to the family Coronaviridae at high proliferation accuracy. In addition, it is possible to provide the animal model infected with the virus belonging to the family Coronaviridae that can produce the neutralizing antibody.

### BRIEF DESCRIPTION OF DRAWINGS

FIG. 1 (Panel A) shows an effect of adding trypsin to a virus solution Lot 1. FIG. 1 (Panel B) shows an effect of adding the trypsin to a virus solution Lot 2.

FIG. 2 shows changes in a viral load over time in each individual inoculated with the virus.

FIG. 3 shows results of monitoring a body temperature in each individual inoculated with the virus. Panel A shows the body temperature of CE0202M. Panel B shows the body temperature of CE0324F. Panel C shows the body temperature of CE1242F. Panel D shows a body temperature change of the CE0202M. Panel E shows a body temperature change of the CE0324F. Panel F shows a body temperature change of the CE1242F.

FIG. 4 (Panel A) shows results of monitoring body weight of each individual inoculated with the virus. FIG. 4 (Panel B) shows blood oxygen saturation of each individual inoculated with the virus. Marker ▪ indicates a result of individual ID: CE0202M. Marker ▴ indicates a result of individual ID: CE0324F. Marker ● indicates a result of individual ID: CE1242F.

FIG. 5 (Panel A) shows a chest radiograph of the individual ID: CE0324F at the time of the inoculation (Day 0). FIG. 5 (Panel B) shows a chest radiograph of the individual ID: CE0324F three days after the inoculation (Day 3). FIG. 5 (Panel C) shows a chest radiograph of the individual ID: CE0324F seven days after the inoculation (Day 7). FIG. 5 (Panel D) shows a macroscopic image of a lung taken at necropsy from the individual ID: CE0202M. FIG. 5 (Panel E) shows a macroscopic image of a lung taken at necropsy from the individual ID: CE0324F. FIG. 5 (Panel F) shows a macroscopic image of a lung taken at necropsy from the individual ID: CE1242F.

FIG. 6 (Panel A) shows an image of an HE-stained peripheral lung tissue of the individual ID: CE0324F. FIG. 6 (Panel B) shows an image of an HE-stained peribronchial lung tissue of the individual ID: CE0324F. FIG. 6 (Panel C) shows an image of an HE-stained salivary gland tissue of the individual ID: CE0324F. FIG. 6 (Panel D) shows an image of an HE-stained lung tissue of the individual ID: CE0202M. FIG. 6 (Panel E) shows an image of an HE-stained thrombus part in the lung tissue of the individual ID: CE0202M. FIG. 6 (Panel F) shows an image of an HE-stained lung tissue including bronchi of the individual ID: CE0324F. FIG. 6 (Panel G) shows an image of an immunostained angiotensin-converting enzyme 2 in a lung tissue. FIG. 6 (Panel H) shows an image of an immunostained angiotensin-converting enzyme 2 in a bronchial tissue. FIG. 6 (Panel I) shows an image of an immunostained angiotensin-converting enzyme 2 in a kidney tissue.

FIG. 7 shows a biochemical data for each individual inoculated with the virus. Panel A shows total bilirubin concentration. Panel B shows alanine aminotransferase (ALT) values. Panel C shows alkaline phosphatase (ALP) values. Panel D shows urea nitrogen (BUN) levels. Panel E shows creatinine levels. Panel F shows amylase levels. Panel G shows glucose levels. Marker ▪ indicates a result of the individual ID: CE0202M. Marker ▴ indicates a result of the individual ID: CE0324F. Marker ● indicates a result of the individual ID: CE1242F.

FIG. 8 shows a hematological data for each individual inoculated with the virus. Panel A shows white blood cell counts. Panel B shows granulocyte counts. Panel C shows lymphocyte counts. Panel D shows monocyte counts. Panel E shows platelet counts. Marker ▪ indicates a result of the individual ID: CE0202M. Marker ▴ indicates a result of the individual ID: CE0324F. Marker ● indicates a result of the individual ID: CE1242F.

FIG. 9 shows concentrations of cytokines and chemokines in plasma of each individual inoculated with the virus. Marker ▪ indicates a result of the individual ID: CE0202M. Marker ▴ indicates a result of the individual ID: CE0324F. Marker ● indicates a result of the individual ID: CE1242F.

FIG. 10 shows results of measurement of cytokine-producing cells by ELISPOT using cells collected from each individual inoculated with the virus. Panel A shows results of the individual ID: CE0202M. Panel B shows results of the individual ID: CE0324F. Panel C shows results of the individual ID: CE1242F. Marker ▪ indicates interferon-γ-producing cells. Marker ● indicates IL-2-producing cells.

FIG. 11 shows neutralizing activity against the virus in the plasma of each individual inoculated with the virus.

FIG. 12 shows results of a qualitative test that examined whether or not the IgG and IgM, which react with the SARS-CoV-2, are present in the plasma of individuals inoculated with the virus.

FIG. 13 (Panel A) shows IgG antibody titers against SARS-CoV-2 S1 protein. FIG. 13 (Panel B) shows IgG antibody titers against SARS-CoV-2 N protein. Marker ▪ indicates a result of the individual ID: CE0202M. Marker ▴ indicates a result of the individual ID: CE0324F. Marker ● indicates a result of the individual ID: CE1242F.

### DESCRIPTION OF EMBODIMENTS

### I. Virus Proliferation Method

One embodiment of the present invention relates to a proliferation method for in vitro proliferating a virus. The proliferation method comprises a step for culturing kidney-derived cells, which have been contacted with a virus belonging to the family Coronaviridae, with the use of a cell culture medium to which a protease having an optimum pH of from 7 to 9 is added.

As used herein, the virus is not limited as long as it belongs to the family Coronaviridae. For example, the virus belonging to the family Coronaviridae includes severe acute respiratory syndrome coronavirus 2 (SARS-CoV-2), SARS coronavirus, human coronavirus 229E strain, human coronavirus NL63 strain, human coronavirus OC43 strain, human coronavirus HKU1 strain, and the like. The virus belonging to the family Coronaviridae is preferably the SARS-CoV-2 or the SARS coronavirus, and more preferably the SARS-CoV-2. The SARS-CoV-2 may include a virus having a genome sequence registered under NCBI Reference Sequence: NC_045512.2, SARS-CoV-2 JP/TY/WK-521/2020 (GenBank: LC522975.1), and a virus having a genome sequence that is 70% or more, 80% or more, 85% or more, 90% or more, 93% or more, 95% or more, 98% or more, 99% or more, or 99.5% or more identical to that of these viruses.

The virus belonging to the family Coronaviridae (hereinafter, sometimes simply abbreviated as a “virus”) can be proliferated in vitro using the kidney-derived cell. The kidney-derived cell is not particularly limited. A cell derived from a kidney tissue of a mammal such as a human, a monkey and a dog can be used. The kidney-derived cell is preferably a cell expressing angiotensin-converting enzyme 2 (ACE2). The kidney-derived cell may be a primary cultured cell or a cell established as a cultured cell line. The cultured cell line includes, for example, Vero cell, Vero E6 cell, and the like. The Vero E6 cell is preferred.

A method for contacting the virus with the kidney-derived cell is not limited as long as the virus can contact the surface of the kidney-derived cell. For example, the virus is suspended in a liquid such as Hanks Balanced Salt Solution (HBSS), PBS, a basal medium described later, or a cell culture medium to prepare a virus suspension, and the virus suspension is applied to the kidney-derived cells adhered to a plate or a flask to allow the virus to be in contact with the kidney-derived cells. The virus to be suspended in the liquid may be contained in a biological sample such as a nasal swab, an oral swab, a bronchial swab and a tracheal swab, or suspension of the swab; blood; pleural effusion; sputum. Also, the virus to be suspended in the liquid may be contained in a culture supernatant or the like. The biological sample is preferably contacted with the kidney-derived cell within, for example, 24 hours after collection.

The contact between the virus and the kidney-derived cell is preferably carried out by, for example, applying the virus suspension to the kidney-derived cells and then incubating at 20° C. to 28° C. for about 30 to 90 minutes. When using the plate, the incubation is preferably carried out by standing, and when using the flask, the incubation is preferably carried out using a seesaw shaker or the like while tilting the flask at intervals of about 3 to 7 minutes.

The kidney-derived cell contacted with the virus can be cultured in a culture environment corresponding to culture conditions for the kidney-derived cell. A culture temperature is, for example, 33° C. to 37° C., and the culture can be performed in the presence of about 5% carbon dioxide gas in a wet state.

For example, as the cell culture medium, a medium obtained by adding bovine serum albumin at a final concentration of about 0.05% to 0.2% to the basal medium such as MEM medium, D-MEM medium, or α-MEM medium can be used. If necessary, an antibiotic such as streptomycin at a final concentration of 50 μg/mL to 150 μg/mL and penicillin at a final concentration of about 50 unit/mL to 150 unit/mL may be added as long as it does not interfere with the proliferation of the virus.

In addition, a protease can be added to the cell culture medium. The protease is not limited as long as it exerts activity at pH of the culture environment for the kidney-derived cell. For example, an optimum pH for the protease can be pH 7 to pH 9. Preferably, the protease is an endopeptidase. Also, the protease is preferably serine protease or threonine peptidase. The protease includes, preferably, trypsin, chymotrypsin, acetylated trypsin, and the like. The protease is, more preferably, trypsin.

The protease may be added to the cell culture medium at a final concentration of about 5 units/mL to 25 units/mL as an enzyme activity value. Preferably, the final concentration is about 10 unit/mL to 15 unit/mL. For example, quality of the trypsin can be determined according to JIS K 0605:2000. As to enzymes other than the trypsin, enzyme activity thereof equivalent to the trypsin can be determined by calculating relative activity to the trypsin.

Here, in the present description, the phrase “add a protease” means that the protease is externally added to the cell culture medium or the like outside the cell.

The kidney-derived cell contacted with the virus can be cultured in the cell culture medium under the culture environment described above for about 2 to 7 days, preferably about 6 days.

When the virus proliferates in the kidney-derived cell, the cell is degenerated. The degeneration of the cell is intended a state in which the cell is dying or has died. The degeneration of the cell can be determined by phase-contrast microscopy using an inverted microscope or the like. The degeneration of the cell can also be determined by staining using crystal violet.

Further, the method for proliferating the virus may comprise a step for collecting the proliferated viruses from the culture. The viruses can be collected, for example, by collecting a culture supernatant from the plate or the flask as a culture and collecting cell residues. Alternatively, after collecting the cells, the virus may be collected by disrupting the cells with ultrasonic or the like and removing cell residues from the disrupted cell suspension. A deoxyribonuclease may be added to the disrupted cell suspension as needed. The cell residues can be removed by centrifuging at about 1,400 r.p.m for, for example, 5 to 10 minutes to remove a precipitate. In addition, a supernatant may be collected by centrifugation, followed by filtration of the supernatant to remove cell residues.

### II. Method for Producing Neutralizing Antibody

One embodiment of the present invention relates to a method for producing a neutralizing antibody to the virus belonging to the family Coronaviridae. The production method may comprise following first to seventh steps.

### (i) First Step

A first step is a step for inoculating a monkey belonging to the Macaca with the virus belonging to the family Coronaviridae and/or the part thereof serving as an antigen.

The monkey belonging to the Macaca may include a Macaca fascicularis, a Macaca mulatta, a Macaca fuscata, a Macaca cyclopis and the like. The monkey belonging to the Macaca is preferably the Macaca fascicularis or the Macaca mulatta, more preferably the Macaca fascicularis.

Here, sex and age of the monkey are not particularly limited. For example, it is preferable to use a monkey of about 3 to 15 years of age.

The virus belonging to the family Coronaviridae is as described in the above I., and hence the above description is incorporated herein by reference. The part of the virus belonging to the family Coronaviridae is intended to be a part of components constituting the virus and have antigenicity. For example, the virus belonging to the family Coronaviridae has an envelope, in which a spike (S) protein, an envelope (E) protein, and a membrane (M) protein are present, and genomic RNA. In addition, an N protein that is an RNA-binding protein is bound to genomic RNA. These proteins or parts thereof can be the antigen. Thus, antigen can include the entire virus as well as at least a portion of the S, E, M and N proteins.

The inoculation of the antigen to the monkey belonging to the Macaca can be performed by a known method. For example, when inoculating the virus, the virus may be inoculated onto the conjunctiva, the nostril, the oral cavity, the trachea, or the like of the monkey by using a pipette or catheter, so that the total amount of the virus to be inoculated per individual is 1×10⁵ to 1×10⁸ TCID₅₀. The inoculation of the antigen is preferably performed under anesthesia.

When a portion of a virus is used as an antigen, it may be inoculated onto the conjunctiva, nostril, oral cavity, or trachea, as well as into blood, subcutaneous, or the like.

### (ii) Second Step

In a second step, a blood sample is collected from the monkey inoculated with the antigen. The blood sample is preferably collected, for example, 14 to 28 days after the inoculation with the antigen. The blood sample may include whole blood, serum, and plasma. The blood sample is preferably the plasma. An anticoagulant used when collecting the plasma is not particularly limited. For example, as the anticoagulant, heparin can be used, although it is not particularly limited.

### (iii) Third Step

In a third step, the blood sample collected in the second step is mixed with the virus belonging to the family Coronaviridae (hereinafter sometimes simply referred to as a “virus”). If an antibody to the inoculated antigen is produced by inoculating the antigen in the first step, the produced antibody binds to the virus when mixed with the blood sample. The third step is not limited as long as it is performed in an environment where the antibody can bind to the virus. For example, it can be performed at 20° C. to 37° C. for about 15 minutes to 1 hour. Here, the blood sample is preferably added with Receptor Destroying Enzyme (RDEII, Denka Seiken, Tokyo, Japan) or the like and treated at 35° C. to 37° C. overnight. It is expected that such treatment prevents proteins in the blood sample from binding to proteins on the surface of the virus that are used by the virus to adhere to the cells. It is preferable to inactivate a complement contained in the blood sample in advance. A method for inactivating the complement is known. For example, the complement can be inactivated by incubating the blood sample at about 56° C. for about 1 hour.

In addition, the blood sample may be diluted with the liquid for suspending the virus described in the above I. The blood sample can be diluted about 8 to 100,000 times. The virus fluid to be mixed with the diluted blood sample has a virus concentration of, for example, about 4,000 TCID₅₀/mL, and the blood sample or each diluted blood sample and the virus fluid are mixed in equal amounts.

### (iv) Fourth Step

A fourth step is a step for bringing the virus belonging to the family Coronaviridae mixed with the blood sample in the third step into contact with the kidney-derived cell. This step is the same as the step for bringing the virus into contact with the kidney-derived cell described in the above I., and hence the description in the above I. is incorporated herein by reference.

### (v) Fifth Step

In a fifth step, the kidney-derived cells, which have been contacted with the virus belonging to the family Coronaviridae in the fourth step, are cultured with the use of the cell culture medium to which the protease having the optimum pH of from 7 to 9 is added. This step is the same as the step for culturing the kidney-derived cell which has been contacted with the virus described in the above I., and hence the description in the above I. is incorporated herein by reference.

### (vi) Sixth Step

In a sixth step, a degeneration state of the kidney-derived cell cultured in the fifth step is evaluated. The degeneration of the kidney-derived cell infected with the virus is as described in the above I. Evaluating the degeneration state is intended to evaluate whether the kidney-derived cell is degenerated or not. The evaluation is performed, as described in the above I., for example, by determining the degeneration state of the cell by observing cell morphology with the inverted microscope or the like, or by staining using the crystal violet. The evaluation of whether the kidney-derived cell is degenerated or not can be performed 2 to 7 days, preferably 6 days after the contact with the virus.

### (vii) Seventh Step

In a seventh step, it is determined that a neutralizing antibody is produced when there is no denaturation of the kidney-derived cell or a degree of the denaturation of the kidney-derived cell is low in the evaluation of the sixth step. The degree of the denaturation can be represented, for example, by a neutralization titer. For example, the neutralization titer can be represented as a dilution ratio at which a cytopathogenic effect was observed in the well. The cytopathogenic effect can be represented, for example, as a value indicating a dilution ratio at which the number of the viruses infecting the cells is reduced to a predetermined percentage, or as a value indicating a dilution ratio at which the number of plaques in the well is reduced to the predetermined percentage. The predetermined percentage can be selected, for example, within the range of 10% to 60%, and the range can be optionally set, for example, from 10%, 20%, 30%, 40%, 45%, 50%, 55%, and 60%. The predetermined percentage is preferably in the range of 40% to 55%, more preferably 50%. The higher the neutralization titer, the lower the degree of the degeneration of the kidney-derived cell. When the neutralization titer is low or when the virus is not neutralized, the degree of the degeneration of the kidney-derived cell is high. In addition, for example, when the kidney-derived cell which has been contacted with the virus not mixed with the blood sample is used as a positive control, it can be determined that the neutralizing antibody is produced if the degree of the degeneration of the kidney-derived cell which has been contacted with the virus mixed with the blood sample is lower than that of the positive control.

### (viii) Another Embodiment

Another embodiment of the method for producing the neutralizing antibody relates to a method for producing the neutralizing antibody to the SARS-CoV-2 comprising a step for inoculating the SARS-CoV-2 and/or the part thereof serving as the antigen into the Macaca fascicularis. The step for inoculating the SARS-CoV-2 and/or the part thereof serving as the antigen into the Macaca fascicularis corresponds to the first step of the above II., and hence the descriptions in the first step of the above II. and the above I., which is incorporated in the first step of the above II., are incorporated herein by reference for an explanation of a term.

### III. Method for Producing Infection Model of SARS-CoV-2

One embodiment of the present invention relates to a method for producing an infection model of the SARS-CoV-2. The production method may comprise the following Steps I to III.

### (i) Step I

Step I is a step for culturing the kidney-derived cells, which has been contacted with the SARS-CoV-2, with the use of the cell culture medium to which the protease having the optimum pH of from 7 to 9 is added. This step is the same as the step for bringing the virus into contact with the kidney-derived cell described in the above I., and hence the description in the above I. is incorporated herein by reference.

### (ii) Step II

Step II is a step for collecting the SARS-CoV-2 from the culture of the kidney-derived cells. This step is the same as the step for collecting the virus from the culture of the kidney-derived cells described in the above I., and hence the description in the above I. is incorporated herein by reference.

(iii) Step III

Step III is a step for inoculating the collected SARS-CoV-2 and/or a part thereof to the Macaca fascicularis. The description of the first step of the above II. is incorporated herein for the part of the SARS-CoV-2. The step for inoculating the SARS-CoV-2 and/or the part thereof to the Macaca fascicularis corresponds to the first step of the above II., and hence descriptions in the first step of the above II. and the above I., which is incorporated in the first step of the above II., are incorporated herein by reference for an explanation of a term.

### (iv) Step IV

A step for determining whether or not infection has been occurred in the Macaca fascicularis inoculated with the SARS-CoV-2 and/or the part thereof may be comprised as Step IV. Whether or not the infection has been occurred can be determined by body temperature monitoring, chest radiography, necropsy, and histopathology.

### Examples

The present invention will be described in more detail below with reference to examples. However, the present invention should not be construed as being limited to the examples. In addition, following experiments were conducted strictly according to Guidelines for the Husbandry and Management of Laboratory Animals of Research Center for Animal Life Science at Shiga University of Medical Science, Standards relating to the Care and Keeping and Reducing Pain of Laboratory Animals of the Ministry of the Environment, and Fundamental Guidelines for Proper Conduct of Animal Experiment and Related Activities in Academic Research Institutions under the jurisdiction of the Ministry of Education, Culture, Sports, Science and Technology. An experimental protocol was approved by the animal care and use committee of Shiga University of Medical Science (authorization number: 2020-4-2).

In addition, an inoculation experiment using a virus was performed at a biosafety level 3 facility at the Research Center for Animal Life Science, Shiga University of Medical Science.

### I. Determination of Proliferation and Titer of Virus

i. Virus Strain

SARS-CoV-2 JP/TY/WK-521/2020 (GenBank Sequence Accession: LC522975; donated by Dr. Masayuki Saijo and Dr. Masaaki Sato of the National Institute of Infectious Diseases (NIID); Matsuyama, S. et al: Proc Natl Acad Sci USA. 2020 Mar. 31; 117(13):7001-7003. doi: 10.1073/pnas.2002589117.) was used as an inoculum. The virus was proliferated twice at NIID using VeroE6/TMPRSS2 cell and once at Shiga University of Medical Science using VeroE6 cell (American Type Culture Collection, Manassas, Va.).

ii. Cell

VeroE6 cells were cultured in MEM medium (Nacalai Tesque, Inc) supplemented with 10% inactivated fetal bovine serum (FBS; Capricorn Scientific GmbH), penicillin (at a final concentration of 100 units/mL), and streptomycin (at a final concentration of 100 μg/mL) (Nacalai Tesque, Inc). The VeroE6 cells were cultured in a flask.

iii. Proliferation and Collection of Virus

The VeroE6 cells adhered to the bottom of the flask were washed twice with Hanks Balanced Salt Solution (HBSS; Nacalai Tesque, Inc) to remove the HBSS. 5 mL of HBSS containing 10 μL of the seed virus solution donated by the NIID (referred to as a diluted virus solution) was added per flask, and the cells were cultured for 1 hour at room temperature while tilting the flask at intervals of 5 minutes so that the diluted virus solution covers the cells.

25 mL of 0.1% bovine serum albumin (BSA)-added MEM medium was added per flask, and 60 μL of 0.25% trypsin solution was added per flask (at a final trypsin concentration of 5 μg/mL). The VeroE6 cell which has been contacted with the diluted virus solution was cultured at 37° C. in the presence of 5% carbon dioxide gas. Cell morphology was observed with an inverted microscope.

After the VeroE6 cells died, a culture supernatant was collected from the flask, and centrifuged at 1,400 r.p.m. for 5 minutes to collect a supernatant. The collected supernatant was further filtered with a bottle top filter to obtain a filtrate as a virus solution.

iv. Quantitative Determination of Virus Titer

The VeroE6 cells were plated in a 96-well plate and cultured until the cells reach confluence. The virus solution collected in the above I. iii. was subjected to ten-fold serial dilutions with the 0.1% BSA-added MEM medium. After removing the medium, 100 μL each of the undiluted virus solution and the diluted virus solution were added to wells containing the VeroE6 cells, and allowed to stand at 37° C. for 1 hour. A culture medium was prepared by adding 20 μL of 0.25% trypsin solution to 10 mL of the 0.1% BSA-added MEM medium used in the above I. i., and 100 μL of the culture medium was added to each well. After culturing for 6 days at 37° C. in the presence of 5% carbon dioxide gas, the presence or absence of cell degeneration was determined under a microscope.

### V. Verification of Effect of Trypsin

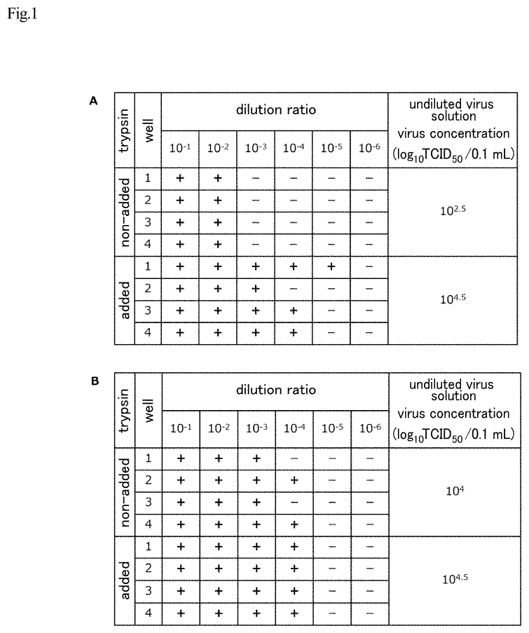

In order to demonstrate that the addition of trypsin is effective for in vitro proliferating the virus, a virus titer when the virus was proliferated in the presence of the trypsin was compared with a virus titer when the virus was proliferated in the absence of the trypsin. Two types of virus solutions, Lot 1 and Lot 2, were used. A 10-fold serial dilution series was prepared for each Lot and inoculated into VeroE6 cells in 4 wells, respectively. For each series, in the same manner as in the above I. iv., the cells were divided into a trypsin-added group in which the virus was proliferated with the addition of the trypsin and a non-trypsin-added group in which the virus was proliferated without the addition of the trypsin, and the presence or absence of the cell degeneration was observed. The results are shown in FIG. 1. In FIG. 1, “+” means that the cell degeneration was observed by microscopy. “−” means that the cell degeneration was not observed by the microscopy. As a result, in the Lot 1, the trypsin-added group had a 100 times higher titer than the non-trypsin-added group (FIG. 1A). In the Lot 2, the trypsin-added group had an about 3 times higher (=10° 5) titer than the non-trypsin-added group (FIG. 1B). In addition, the addition of the trypsin reduced lot-to-lot variation.

From this, it is considered that the addition of a protease is preferable for in vitro proliferating virus.

### II. Virus Infection Experiment

i. Macaca fascicularis

A 15-year-old female Macaca fascicularis (individual ID: CE0324F), a 10-year-old female Macaca fascicularis (individual ID: CE1242F) and a 15-year-old male Macaca fascicularis (individual ID: CE0202M) born at Shiga University of Medical Science were used. The individuals used in this study did not have herpes B virus, hepatitis E virus, Mycobacterium tuberculosis, Shigella, Salmonella, and Entamoeba histolytica.

All treatment were performed under ketamine/xylazine anesthesia. Each individual was placed in a cage and reared. CMK-2 (CLEA Japan, Inc., Tokyo, Japan) was fed as animal feed once daily after recovery from the anesthesia. Drinking water was provided ad libitum. The rearing environment was humidity (39 to 61%), temperature (24 to 26° C.), and a light-dark cycle of 12-hour light (turn the light on at 8:00 am) and 12-hour dark (turn the light off at 8:00 pm).

For monitoring the body temperature, each individual was implanted with a telemetry probe (M00, Data Sciences International, Saint Paul, Minn.) in the peritoneal cavity or subcutaneously under anesthesia with ketamine/xylazine administration and isoflurane inhalation two weeks prior to virus inoculation.

Under the ketamine/xylazine anesthesia, two cotton swabs (Eiken Chemical, Tokyo, Japan) were used to collect conjunctival, nasal, oral, and tracheal fluid samples. Then, the cotton swabs were immersed in 1 mL of MEM medium (Nacalai Tesque, Inc) containing 0.1% bovine serum albumin (BSA) and antibiotics. Bronchial samples were collected using a bronchoscope (MEV-2560, MACHIDA Endoscope Co., Ltd., Tokyo, Japan) and a cytology brush (BC-203D-2006, Olympus Corporation, Tokyo, Japan). Samples were collected on days 0, 1, 3, 7, 10, 14, 17, 21, 24, and 28. Day 0 is the day of the virus inoculation.

Chest radiographs were obtained using I-PACS system (Konica Minolta Inc., Tokyo, Japan) and PX-20BT mini (Kenko Tokina Corporation, Tokyo, Japan).

ii. Infection Experiment

The virus was inoculated into the conjunctiva (0.05 mL×2), nostril (0.5 mL×2), oral cavity (0.9 mL) and trachea (5 mL) of each individual under the ketamine/xylazine anesthesia by a pipette and a catheter so that the total amount of the virus inoculated per individual was 2.2×106 TCID₅₀.

Swabs and tissue samples were taken to evaluate proliferation of the virus in the body of the individuals. Tissues were disrupted to prepare homogenate samples (10% w/v). Serial dilutions of swabs and homogenate samples were prepared and each dilution was inoculated into confluent VeroE6 cells. The VeroE6 cells were cultured in MEM medium supplemented with 0.1% BSA, penicillin, streptomycin, gentamicin (50 μg/mL) (FUJIFILM Corporation, Tokyo, Japan), and trypsin (5 μg/mL) (Nacalai Tesque, Inc). The cytopathogenic effect of the virus was observed under the microscope 6 days after the inoculation.

iii. Virus Neutralization Test

Blood was collected from the individuals inoculated with the virus on days 0, 7, 10, 14, 17, 21, 24, and 28, and plasma was separated. Receptor Destroying Enzyme (RDEII, Denka Seiken, Tokyo, Japan) was added to the plasma samples and treated at 37° C. overnight. It was then incubated at 56° C. for 1 hour to inactivate a complement. The plasma after the inactivation treatment was serially diluted 8-fold, 16-fold, 32-fold, 64-fold, 128-fold, 256-fold, 512-fold, 1024-fold, 2048-fold, 4096-fold, and 8192-fold with MEM medium supplemented with 0.1% BSA, and 120 μL of the diluted plasma sample and 120 μL of the virus diluted solution (4,000 TCID₅₀/mL) were mixed and incubated for 30 minutes at room temperature. Then, the mixture of the plasma sample and the virus was added to Vero E6 cells subjected to monolayer culture in a 96-well plate and incubated for 1 hour. After completion of the incubation, MEM medium supplemented with 0.1% BSA and 5 μg/mL trypsin was added to each well. After adding the medium, the cells were cultured at 37° C. for 6 days, and then wells in which the presence of cytopathogenicity was observed were counted. One diluted plasma sample was tested using 4 wells. The neutralization titer is represented as a dilution ratio at which the cytopathogenic effect was observed in 50% of the wells.

iv. Detection of Antibody to SARS-CoV-2

Plasma was collected from the individuals inoculated with the virus. COVID-19 IgG/IgM immunodetection kit (Novus Biologicals USA, Centennial Co.) was used to measure IgG and IgM specific for SARS-CoV-2 S protein and SARS-CoV-2 N protein, respectively.

v. Detection of Cytokine-Producing Cells by ELISPOT

Blood was collected from the individuals inoculated with the virus, and mononuclear blood cells were separated using Ficoll solution. The cells were centrifuged, suspended in a cell banker cell cryopreservation medium, and then stored at −80° C. until use. ELISPOT plates coated with an anti-IFN-γ antibody, an anti-IL-2 antibody (Cellular Technology Limited, Shaker Heights, Ohio) were used in experiments. Thawed cells were plated on each well of the ELISPOT plate at 5×10⁵ cells/well and incubated with a peptide pool of SARS-CoV-2 N protein (PepTivator, Miltenyi Biotech) in the presence of an anti-CD28 antibody for 1 day. The number of cytokine-producing cell spots was counted according to manufacturer's instructions.

vi. Blood Cytokine and Biochemical Analysis

Levels of cytokines/chemokines in plasma in the individuals inoculated with the virus were measured using Milliplex MAP non-human primate cytokine panel and Luminex 200 (Millipore Corp., Billerica, Mass.) according to manufacturer's instructions. Analysis of blood biochemistry and blood counts were measured using VetScan VS2 and VetScan HM2 (Abaxis, Inc., Union City, Calif.), respectively.

vii. Histopathological Examination and Detection of Viral Antigen by Immunohistochemical Staining

For histopathological examination, the tissue taken at the necropsy was immersed in 10% formalin neutral buffer solution and fixed. The fixed tissue was embedded in paraffin and sliced in a thickness of 3 μm to prepare a section according to a conventional method. The sections were stained with hematoxylin and eosin (H&E) and observed under a light microscope. For immunohistochemical staining, ACE-2 Antibody (aa18-740) LS-C122669 (LifeSpan Biosciences) was used at 2 μg/mL as a primary antibody. Histofine Simple Stain MAX-PO(G) (Nichirei Corporation; Code: 414161) was used as a secondary antibody.

viii. Detection of IgG Antibody to 51 Protein and IgG Antibody to N Protein

Plasma was collected from each individual, and IgG antibodies to the SARS-CoV-2 S1 protein (left) and N protein (right) were measured using the MILLIPLEX® SARS-CoV-2 Antigen Panel 1 IgG antibody measurement kit. A measurement method followed the protocol attached to the kit.

ix. Results
(i) Major Proliferation of SARS-CoV-2 in Nasal Cavity and Oral Cavity of Macaca fascicularis

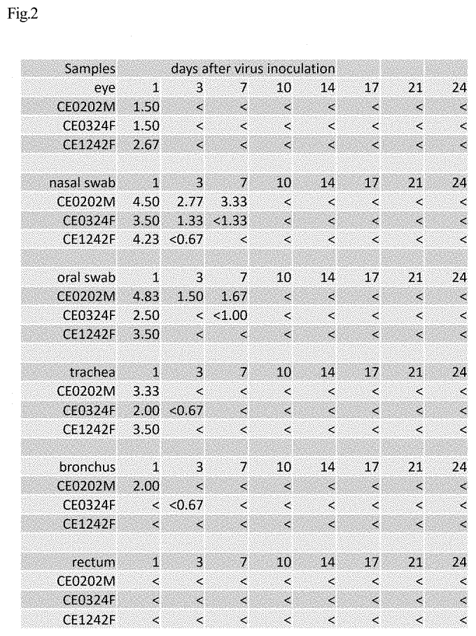

Swabs were sampled from the conjunctiva, nasal cavity, oral cavity, and trachea of the individuals inoculated with the virus using the cotton swab, and proliferation of the virus in each individual was examined. The virus was detected in swabs sampled from the conjunctiva, nasal cavity, oral cavity, and trachea of 3 individuals on the day 1 (the next day after the virus inoculation) (FIG. 2). The virus was detected in swabs sampled from the nasal cavity and oral cavity of 2 individuals (CE0202M and CE0324F) until the day 7. No virus was detected in swabs sampled after the day 10 and in tissue samples taken at the necropsy on the day 28.

(ii) Clinical Diagnosis of Disease in Individuals Inoculated with SARS-CoV-2

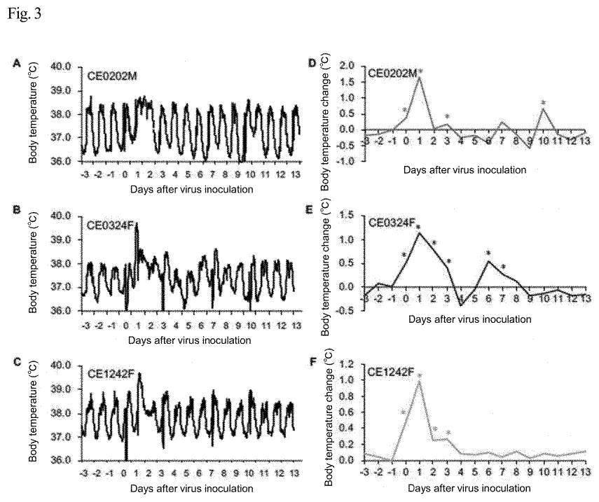

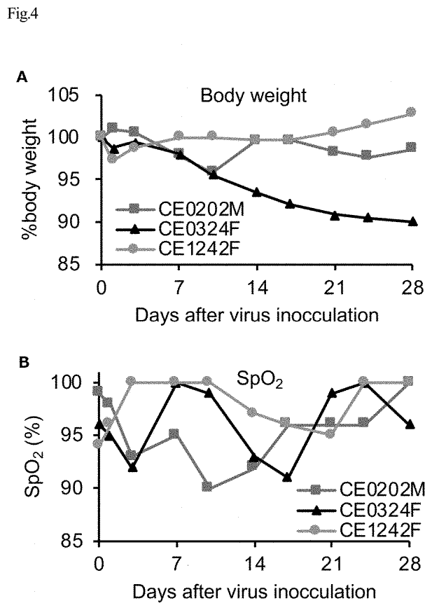

Individuals after being subjected to the virus inoculation showed clinical symptoms. An increase in body temperature was shown in all individuals between the day 1 and day 2 after the virus inoculation (FIGS. 3A-C). On the day 1, the body temperature of two individuals (CE0324F and CE1242F) exceeded 39.5° C. On the other hand, an increase in the body temperature was shown in another individual (CE0202M) only at night. Since daytime body temperature was affected by the anesthesia as shown in FIGS. 3A-C, an average body temperature from 8:00 μm to 8:00 am on the next day was calculated for each individual based on the data shown in FIGS. 3A-C. For example, the body temperature on the day 0 in FIGS. 3D-F shows the average body temperature from 8:00 pm on the day 0 after the virus inoculation to 8:00 am on the day 1 after the virus inoculation. The average body temperature of each day was compared with that of the previous day (day −1), and changes resulting from the comparison are shown in FIGS. 3D-F. “*” in the figure indicates that there was a significant difference (p<0.05 in Student's t-test) in the comparison with the previous day. In addition, FIG. 4 shows the results of monitoring the body weight of each individual. The body weight of the CE0324F decreased by 10% on the day 28 due to anorexia (FIG. 4A). From the above results, infection with the inoculated SARS-CoV-2 was occurred in each individual. However, it is shown that the SARS-CoV-2 causes clinical signs in each individual after infection, while symptoms caused by the SARS-CoV-2 vary from individual to individual. In addition, as for the individuals which had fever, some of them had fever from the day after the infection, indicating that an incubation period under this infection condition was very short.

(iii) Viral Pneumonia in Individuals Infected with SARS-CoV-2

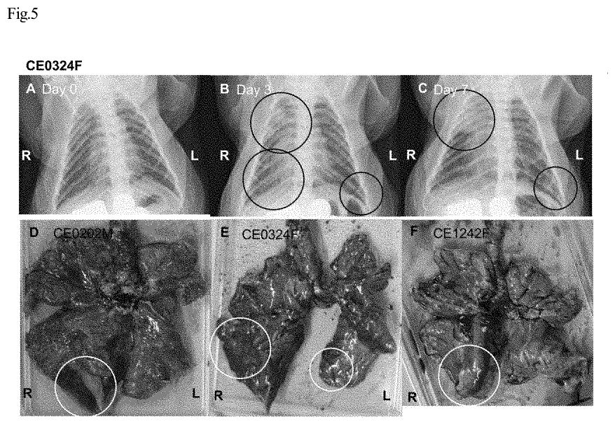

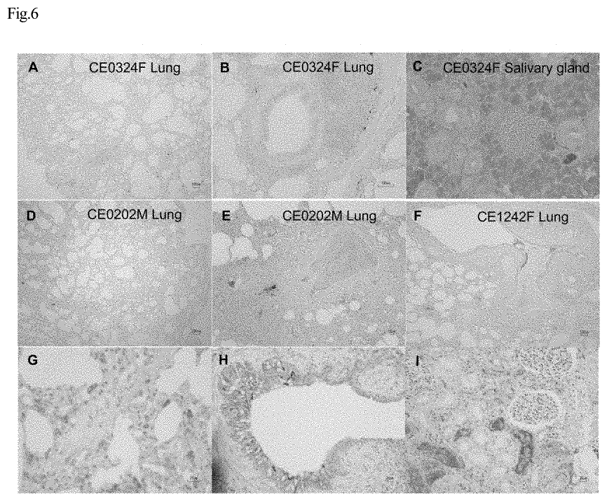

Chest radiography of the individuals inoculated with the virus and histopathological analysis of the tissues taken at the necropsy were performed. Viral pneumonia was observed in the individuals inoculated with the virus. In the CE0324F, ground glass like findings in the lungs were observed in the chest radiograph on the day 3 and the day 7 (FIGS. 5A-C). The pneumonia disappeared 10 days after the virus inoculation. Oxygen saturation (SpO₂) of the individuals inoculated with the virus exceeded 90% during the experiment (FIG. 4B). Macroscopic images of the lungs taken at the necropsy on the day 28 are shown. At the necropsy on the day 28, gross lesions were observed on the surface of the lungs in all individuals inoculated with the virus (FIGS. 5D-F). Specifically, dark red and brown lesions in the CE0324F, bright red lesion in the CE0202M, and small red lesion area in the CE1242F (FIGS. 5D-F) were observed. Histopathological analysis of the lung tissues taken at the necropsy revealed thickening of the alveolar wall accompanied by infiltration of a macrophage in the lungs in the CE0324F (FIGS. 6A-B). Thus, histopathological findings indicated that the macroscopic findings in the lungs of the CE0324F taken at the necropsy (FIG. 5E) were findings of a state in which damaged tissue has been repaired from damage. In a histopathological analysis of the lung tissues taken at the necropsy of the CE0202M, a congestion and thrombus in a pulmonary vessel was observed (FIGS. 6D-E). A bronchus-associated lymphoid tissue (BALT) was formed in the lung tissue of the CE0324F (FIG. 6B). The CE1242F had bronchopneumonia (FIG. 6F).

Lymphocytic infiltration in a submucosal tissue of the trachea of the CE0324F was observed. In addition, as a lesion other than that in a respiratory tissue, localized lymphocytic infiltration was observed in the submandibular gland of the CE0324F.

Angiotensin-converting enzyme 2 (ACE2), which is an entry receptor for the SARS-CoV-2, was detected in alveolar epithelial type II cells in the lung and proximal tubules in the kidney (FIGS. 6G-I). It was suggested that these cells might be infected with the SARS-CoV-2.

(iv) Biochemical Data and Hematological Data of Peripheral Blood of Individuals Inoculated with SARS-CoV-2

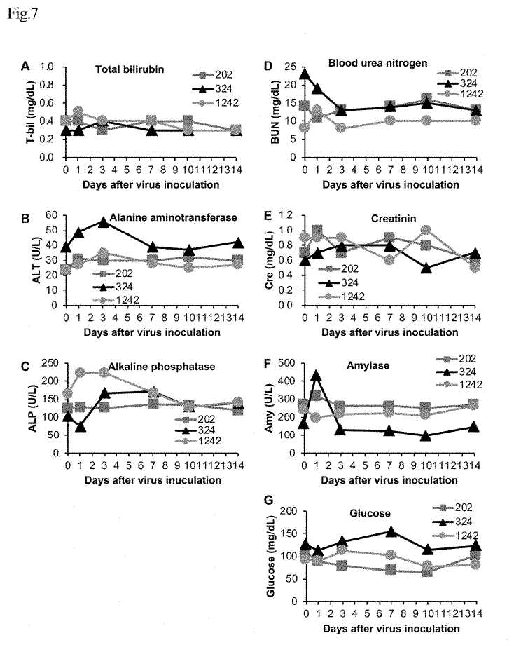

The function of each organ after the SARS-CoV-2 infection was evaluated by biochemical tests. The levels of alanine aminotransferase (ALT) and alkaline phosphatase (ALP) in the plasma of the CE0324F and the CE1242F increased from 1 day to 3 days after the virus inoculation, and returned to the same level as before the inoculation about 7 days after the inoculation. However, these increases did not deviate from a normal range. Total bilirubin in the plasma of these two individuals was also within a normal range, and liver damage was minimal (FIGS. 7A-C). No increases in blood urea nitrogen (BUN) and creatinine were detected, and a renal function was normal after the virus inoculation (FIGS. 4D-E). The CE0324F had a slightly higher blood glucose level than the other individuals. This individual had a transient increase in a plasma amylase level on the day 1 (FIGS. 4F-G). In addition, this individual is the individual in which lymphocytic infiltration into a salivary gland tissue was observed in FIG. 6C.

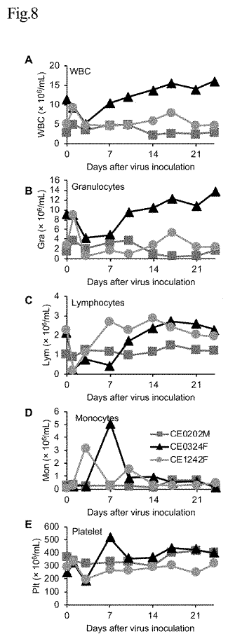

White blood cell counts (WBC) and granulocyte counts (Gra) of the CE0202M and the CD1242F increased transiently on the day 1, whereas white blood cell count of the CE0324F decreased on the days 1 and 3 (FIGS. 8A, B). Lymphocyte counts of the CE0324F and the CE1242F decreased on the day 1 after the virus inoculation and returned to the same level as before the inoculation from the day 7 to the day 10 after the inoculation (FIG. 8C). On the other hand, monocyte counts of the CE0324F and the CE1242F increased on the day 7 and the day 3 after the virus inoculation, respectively (FIG. 8D). Platelet counts of the CE0324F and the CE1242F decreased on the day 3 after the virus inoculation. Thus, two of the three individuals (CE0324F and CE1242F) showed clear changes in blood cell populations.

(v) Immunoreaction of Individuals Inoculated with SARS-CoV-2

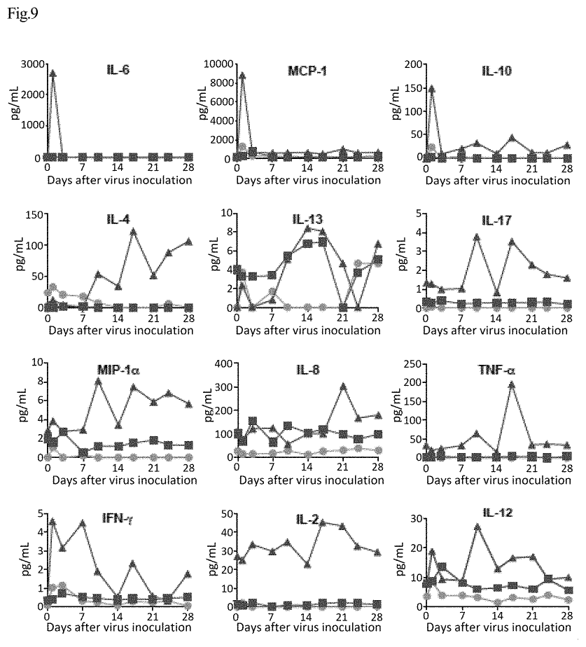

An immune response to the SARS-CoV-2 of the individuals inoculated with the virus was evaluated using the cytokines/chemokines in blood as indicators. Results are shown in FIG. 9.

The CE0324F showed significant changes in cytokine levels in plasma after the inoculation. Specifically, levels of interleukin-6 (IL-6), monocyte chemotactic protein-1 (MCP-1) and IL-10, which are inflammatory cytokines, on the day 1 after the inoculation were higher than before the inoculation. IL-4, IL-13, IL-17 and MIP-1α increased after the day 10 after the inoculation. Changes in the IL-17 and the MIP-1α were small. IL-8 increased on the day 21 after the inoculation and then decreased. Interferon-γ (IFN-γ) and IL-12 increased on the day 1 after the inoculation. The CE1242F had small changes in cytokines. The CE0202M showed transient increases in the IL-12, MIP-1α, IL-8, and IL-13, but the changes were not large.

### (vi) Cytokine-Producing Cells

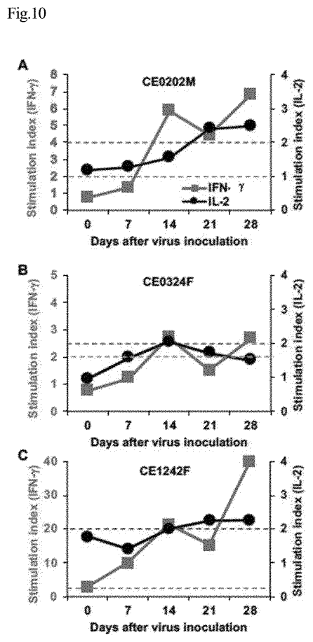

Cytokine-producing cells were detected by ELISPOT. Results are shown in FIG. 10. SARS-CoV-2 N protein peptide-reactive IFN-γ- and IL-2-producing cells increased in the CE0324F from the day 14 after the infection. In the CE0202M, the IFN-γ-producing cells increased from the day 14 after the infection, and the IL-2-producing cells increased from the day 21 after the infection. In the CE1242F, the IFN-γ-producing cells increased from the day 7 after the infection, and the IL-2-producing cells increased from the day 14 after the infection.

(vii) Neutralizing Antibody

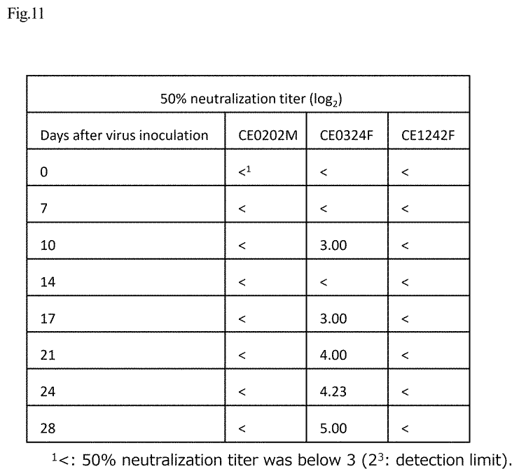

Neutralizing antibody to the SARS-CoV-2 was detected in the CE0324F on the day 10 after the virus inoculation. The neutralizing antibodies were not detected in the other two individuals by day 28 (FIG. 11).

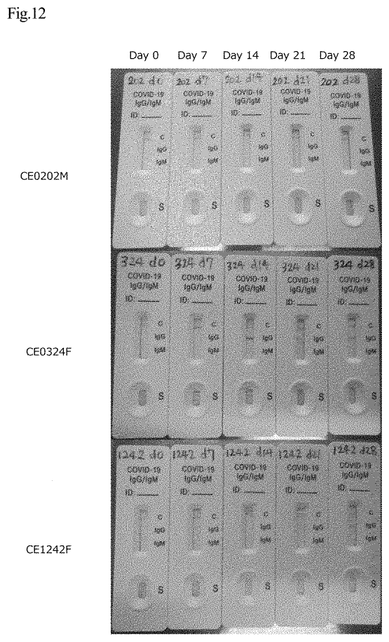

A qualitative test of IgG and IgM reactive to the SARS-CoV-2 in the plasma of the individuals inoculated with the virus was performed using an antibody detection kit for detecting human IgG/IgM against the SARS-CoV-2 S and N proteins. Antibodies to the SARS-CoV-2 were detected after the day 14 in the CE0324F and after the day 21 in the CE1242F (FIG. 12).

From the above results, the pathology of the SARS-CoV-2 varied from individual to individual. However, in some individuals, the neutralizing antibodies were produced in their bodies after the SARS-CoV-2 infection. From this, it was considered that the SARS-CoV-2 can be eliminated immunologically.

(viii) Detection of IgG Antibody to S1 Protein and IgG Antibody to N Protein

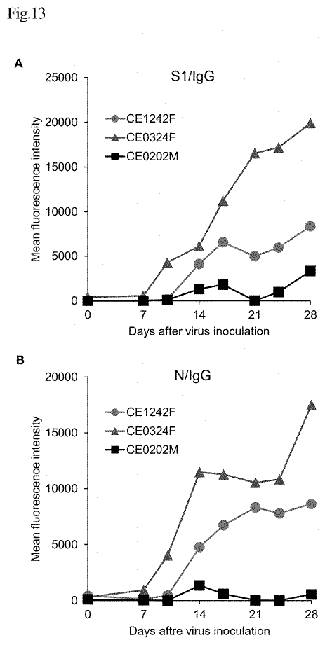

FIG. 13 shows measurement results for each individual. FIG. 13A shows IgG antibody titers against the SARS-CoV-2 S1 protein. FIG. 13B shows IgG antibody titers against the SARS-CoV-2 N protein. Both the IgG against the S1 protein and the IgG against the N protein could be detected in all individuals. The individual (CE0324F) in which the neutralizing antibody was detected in the above ix. (vii) showed a high antibody titer. An antibody titer of the individual (CE1242F) in which the antibody was detected in the above ix. (vii) was moderate. A small amount of IgG was also detected in the individual (CE0202M) in which the antibody was not detected in the above ix. (vii).

1. A method for in vitro proliferating severe acute respiratory syndrome coronavirus 2, comprising a step for culturing monkey kidney-derived cells, which have been contacted with the severe acute respiratory syndrome coronavirus 2, with a use of a cell culture medium to which a protease having an optimum pH of from 7 to 9 is added,
wherein
the protease is at least one selected from the group consisting of trypsin, chymotrypsin, and acetylated trypsin, and
the protease is added into the cell culture medium at a final concentration of 25 units/mL or less as enzyme activity value, and at the range exhibiting the activity of the proteolytic enzyme.

2-4. (canceled)

5. A method for producing a neutralizing antibody to severe acute respiratory syndrome coronavirus 2 comprising,
a first step for inoculating the virus belonging to the severe acute respiratory syndrome coronavirus 2 and/or a part thereof serving as an antigen to a monkey belonging to the Macaca,
a second step for collecting a blood sample from the monkey inoculated with the antigen,
a third step for mixing the blood sample with the virus belonging to the severe acute respiratory syndrome coronavirus 2,
a fourth step for bringing the virus belonging to the severe acute respiratory syndrome coronavirus 2 mixed with the blood sample into contact with monkey kidney-derived cells,
a fifth step for culturing the monkey kidney-derived cells, which have been contacted with the severe acute respiratory syndrome coronavirus 2, with a use of a cell culture medium to which a protease having an optimum pH of from 7 to 9 is added,
a sixth step for evaluating a condition of degeneration of the monkey kidney-derived cells cultured in the fifth step,
a seventh step for determining that neutralizing antibodies are produced when there is no denaturation of the monkey kidney-derived cells or degree of the denaturation of the monkey kidney-derived cells is low in the evaluation of the sixth step,
wherein
the protease is at least one selected from the group consisting of trypsin, chymotrypsin, and acetylated trypsin, and
the protease is added into the cell culture medium at a final concentration of 25 units/mL or less as enzyme activity value, and at the range exhibiting the activity of the proteolytic enzyme.

6. The method for producing the neutralizing antibody according to claim 5, wherein the monkey is a Macaca fascicularis.

7-8. (canceled)

9. A method for producing an infection model of severe acute respiratory syndrome coronavirus 2 comprising:
a step for culturing monkey kidney-derived cells, which have been contacted with the severe acute respiratory syndrome coronavirus 2, with a use of a cell culture medium to which a protease having an optimum pH of from 7 to 9 is added, wherein the protease is at least one selected from the group consisting of trypsin, chymotrypsin, and acetylated trypsin, and the protease is added into the cell culture medium at a final concentration of 25 units/mL or less, and at the range exhibiting the activity of the proteolytic enzyme,
a step for collecting the severe acute respiratory syndrome coronavirus 2 from the culture of the monkey kidney-derived cells,
a step for inoculating the collected the severe acute respiratory syndrome corona virus 2 and/or a part thereof to a Macaca fascicularis.
